(19) United States
(12) Patent Application Publication
(10) Pub. No.: US 2011/0237520 A1
(43) Pub. Date: Sep. 29, 2011

(54) Anticancer Compounds

(75) Inventors: Laura Coello Molinero, Madrid (ES); Rogelio Fernández Rodriguez, Madrid (ES); José Fernando Reyes Benitez, Madrid (ES); Andrés Francesch Solloso, Madrid (ES); Maria del Carmen Cuevas Marchante, Madrid (ES)

(73) Assignee: Pharma Mar, SA, Madrid (ES)

(21) Appl. No.: 13/133,359

(22) PCT Filed: Dec. 18, 2009
(86) PCT No.: PCT/EP2009/067470

§ 371 Date: Jun. 7, 2011

(30) Foreign Application Priority Data
Dec. 19, 2008 (EP) 08382080.3

(51) Int. Cl. A61K 38/12; C07K 7/56; A61P 35/00

(52) U.S. Cl. 514/19.3; 530/323; 514/21.1

(57) ABSTRACT

Compounds of general formula I:

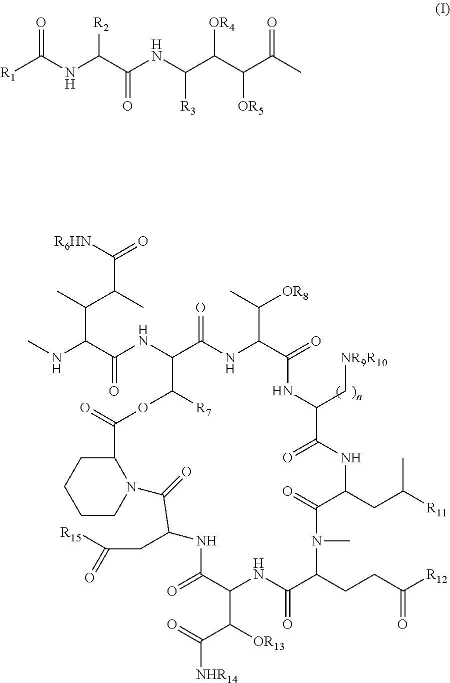

wherein R₁₋R₁₅ and n take permitted meanings for use in the treatment of cancer.

### FIELD OF THE INVENTION

The present invention relates to new anticancer compounds, pharmaceutical compositions containing them and their use as anticancer agents.

### BACKGROUND OF THE INVENTION

Cyclic depsipeptides have emerged as a very important class of bioactive compounds from marine organisms. Several of these cyclic depsipeptides have been disclosed to have cytotoxic, antiviral and/or antifungal properties. Specifically, neamphamide A was disclosed to be isolated from the marine sponge Neamphius huxleyi and showed antiviral activity (Oku et al. J. Nat. Prod. 2004, 67(8), 1407-1411).

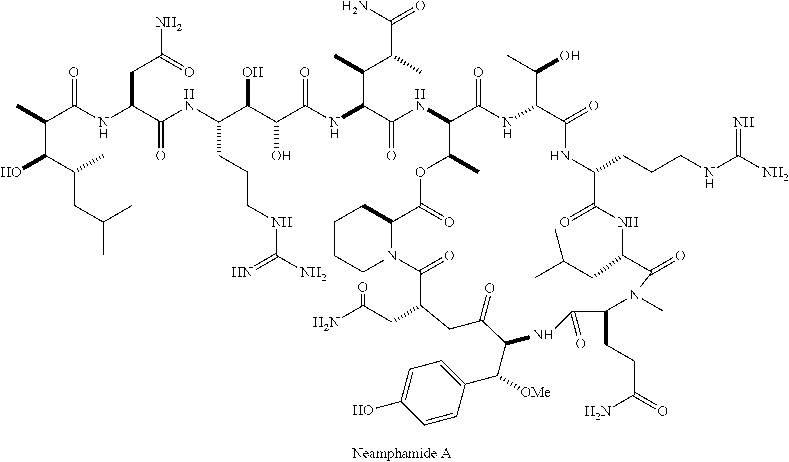

In particular, the anti-HIV activity of neamphamide A was evaluated in a XTT-based cell viability assay using the human T-cell line CEM-SS infected with HIV-1_{RF}. After a 6 day incubation period, neamphamide A effectively inhibited the cytopathic effect of HIV-1 infection with an EC₅₀ of 28 nM.

In 1999, Ford et al. disclosed the isolation of four novel cyclic depsipeptides named papuamides A, B, C, and D from the sponges Theonella mirabilis and Theonella swinhoei. In addition, the synthesis of a diacetate derivative of papuamide A was disclosed (Ford et al. J. Am. Chem. Soc. 1999, 121(25), 5899-5909).

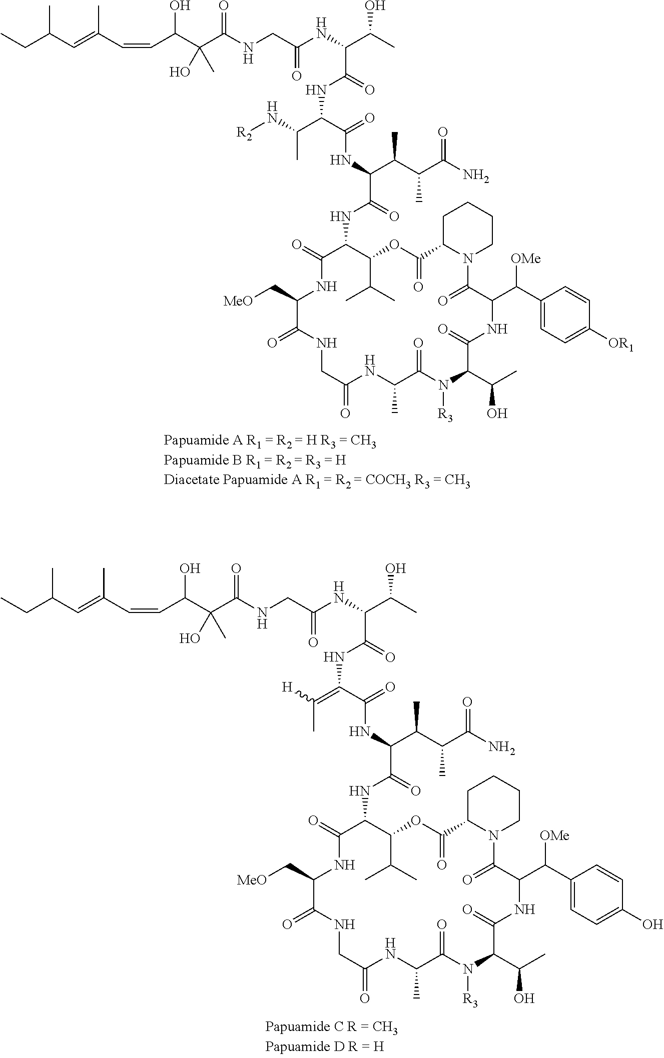

It was found that papuamides A and B inhibited the infection of human T-lymphoblastoid cells by HIV-1_{RF} in vitro with an EC₅₀ of approximately 4 ng/mL. In addition, papuamide A was found to be cytotoxic against a panel of human cancer cell lines with a mean IC₅₀ of 75 ng/mL.

Finally, Zampella et al. also reported further depsipeptides with anti-HIV activity. Specifically, they isolated homophymine A from the sponge Homophymia sp, which exhibited cytoprotective activity against HIV-1 infection with an IC₅₀ of 75 nM in a cell-based XTT assay (Zampella et al. J. Org. Chem. 2008, 73, 5319-5327).

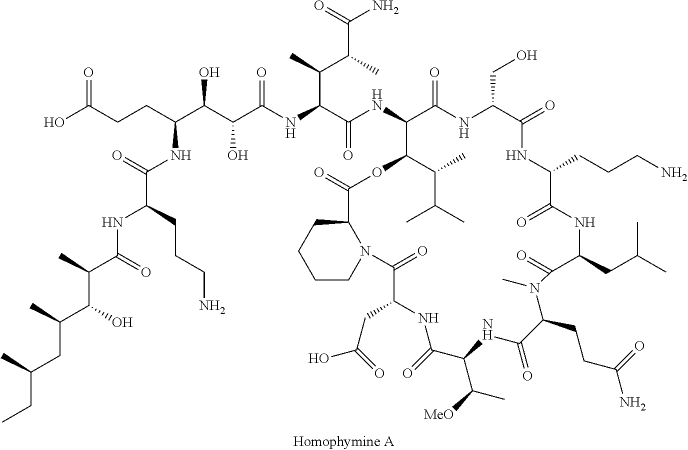

Since cancer is a leading cause of death in animals and humans, several efforts have been and are still being undertaken in order to obtain an anticancer therapy active and safe to be administered to patients suffering from a cancer. The problem to be solved by the present invention is to provide compounds that are useful in the treatment of cancer.

### SUMMARY OF THE INVENTION

In one aspect, the present invention is directed to a compound of general formula I or a pharmaceutically acceptable salt, tautomer, prodrug or stereoisomer thereof

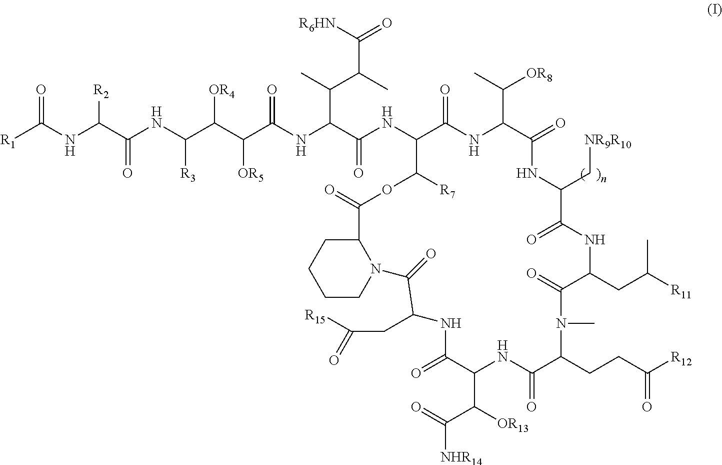

wherein
R₁ is selected from substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl, and substituted or unsubstituted heterocyclic group;
R₂ is selected from hydrogen, —CH₂CONHR₁₆, and —CH(OR₁₇)CONHR₁₈;
R₃ is selected from —CH₂CH₂CONHR₁₉ and —CH(OR₂O)CH₃;
each R₄, R₅, R₈, R₁₇, and R₂₀ is independently selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl;
each R₆, R₁₄, R₁₆, R₁₈, and R₁₉ is independently selected from hydrogen, CORₐ, COORₐ, CONRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₇, R₁₁, and R₁₃ is independently selected from substituted or unsubstituted C₁-C₁₂ alkyl;
each R₉ and R₁₀ is independently selected from hydrogen, CORₐ, COORₐ, CONRₐR_{b}, C(═NRₐ)NRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₁₂ and R₁₅ is independently selected from OR_{c}, NRₐR_{b}, CORₐ, NRₐCONRₐR_{b}, NRₐC(═NRₐ)NRₐR_{b}, halogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
n is 3 or 4;
R_{c} is selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl; and
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group.

In another aspect, the present invention is directed to a compound of formula I, or a pharmaceutically acceptable salt, tautomer, prodrug or stereoisomer thereof, for use as a medicament, in particular as a medicament for treating cancer.

In a further aspect, the present invention is also directed to the use of a compound of formula I, or a pharmaceutically acceptable salt, tautomer, prodrug or stereoisomer thereof, in the treatment of cancer, or in the preparation of a medicament, preferably for the treatment of cancer. Other aspects of the invention are methods of treatment, and compounds for use in these methods. Therefore, the present invention further provides a method of treating a patient, notably a human, affected by cancer which comprises administering to said affected individual in need thereof a therapeutically effective amount of a compound as defined above.

In a yet further aspect, the present invention is also directed to a compound of formula I, or a pharmaceutically acceptable salt, tautomer, prodrug or stereoisomer thereof, for use as an anticancer agent.

In another aspect, the present invention is directed to pharmaceutical compositions comprising a compound of formula I, or a pharmaceutically acceptable salt, tautomer, prodrug or stereoisomer thereof, together with a pharmaceutically acceptable carrier or diluent.

The present invention also relates to the isolation of compounds of formula I from a sponge of the order Lithistida, family Neopeltidae, genus Homophymia, species Homophymia lamellosa Vacelet & Vasseur, 1971, and the formation of derivatives from the isolated compounds.

### DETAILED DESCRIPTION OF THE INVENTION

The present invention relates to compounds of general formula I as defined above.

In these compounds the groups can be selected in accordance with the following guidance:

Alkyl groups may be branched or unbranched, and preferably have from 1 to about 18 carbon atoms. One more preferred class of alkyl groups has from 1 to about 12 carbon atoms; and even more preferably from 1 to about 6 carbon atoms. Alkyl groups having 1, 2, 3, 4 or 5 carbon atoms are particularly preferred. Methyl, ethyl, n-propyl, iso-propyl and butyl, including n-butyl, tert-butyl, sec-butyl and iso-butyl are particularly preferred alkyl groups in the compounds of the present invention. Another preferred class of alkyl groups has from 7 to about 14 carbon atoms; and even more preferably 8, 9, 10, 11, 12, or 13 carbon atoms. As used herein, the term alkyl, unless otherwise stated, refers to both cyclic and noncyclic groups, although cyclic groups will comprise at least three carbon ring members.

Preferred alkenyl and alkynyl groups in the compounds of the present invention may be branched or unbranched, have one or more unsaturated linkages and from 2 to about 18 carbon atoms. One more preferred class of alkenyl and alkynyl groups has from 2 to about 12 carbon atoms; and even more preferably from 2 to about 6 carbon atoms. Alkenyl and alkynyl groups having 2, 3, 4 or 5 carbon atoms are particularly preferred. Another preferred class of alkenyl and alkynyl groups has from 7 to about 14 carbon atoms; and even more preferably 8, 9, 10, 11, 12, or 13 carbon atoms. The terms alkenyl and alkynyl as used herein refer to both cyclic and noncyclic groups, although cyclic groups will comprise at least three carbon ring members.

Suitable aryl groups in the compounds of the present invention include single and multiple ring compounds, including multiple ring compounds that contain separate and/or fused aryl groups. Typical aryl groups contain from 1 to 3 separated and/or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

Suitable heterocyclic groups include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups in the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

The groups above mentioned may be substituted at one or more available positions by one or more suitable groups such as OR′, ═O, SR′, SOR′, SO₂R′, OSO₂R′, OSO₃R′, NO₂, NHR′, N(R)₂, ═N—R′, N(R)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R)₂, CON(R)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list.

Suitable halogen groups or substituents in the compounds of the present invention include F, Cl, Br, and I.

The term “pharmaceutically acceptable salts, prodrugs” refers to any pharmaceutically acceptable salt, ester, solvate, hydrate or any other compound which, upon administration to the patient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts and prodrugs can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts. Trifluoroacetate is one of the preferred pharmaceutically acceptable salts in the compounds of the invention.

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymorphic forms, it is intended that the invention encompasses all such forms.

Any compound that is a prodrug of a compound of formula I is within the scope of the invention. The term “prodrug” is used in its broadest sense and encompasses those derivatives that are converted in vivo to the compounds of the invention. Examples of prodrugs include, but are not limited to, derivatives and metabolites of the compounds of formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6^{th} ed. (Donald J. Abraham ed., 2001, Wiley) and “Design and Applications of Prodrugs” (H. Bundgaard ed., 1985, Harwood Academic Publishers).

Any compound referred to herein is intended to represent such specific compound as well as certain variations or forms. In particular, compounds referred to herein may have asymmetric centres and therefore exist in different enantiomeric or diastereoisomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exists as atropoisomers. All the stereoisomers including enantiomers, diastereoisomers, geometric isomers and atropoisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are amine-imine, amide-imidic acid, keto-enol, lactam-lactim, etc. In addition, compounds referred to herein may exist in isotopically-labelled forms i.e. compounds which differ in the presence of one or more isotopically-enriched atoms. For example, compounds having the present structures except for the replacement of at least one hydrogen atom by deuterium or tritium, or the replacement of at least one carbon by ¹³C- or ¹⁴C-enriched carbon, or the replacement of at least one nitrogen atom by ¹⁵N-enriched nitrogen are within the scope of this invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

In compounds of general formula I, R₁ is preferably selected from substituted or unsubstituted C₁-C₁₈ alkyl and substituted or unsubstituted C₂-C₁₈ alkenyl, which may be branched or unbranched. More preferred alkyl and alkenyl groups, which may be branched or unbranched, are those having from 7 to about 14 carbon atoms; and even more preferably 8, 9, 10, 11, 12, or 13 carbon atoms. It is particularly preferred that the alkyl and alkenyl groups are substituted by one or more suitable substituents, being the substituents preferably selected from OR′, ═O, SR′, SOR′, SO₂R′, SO₃R′, OSO₂R′, OSO₃R′, NO₂, NHR′, N(R)₂, ═N—R′, N(R)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R′)₂, CON(R)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferably, substituents for the above mentioned alkyl and alkenyl groups are selected from OR′, OSO₂R′, OSO₃R′, halogen, OCOR′, OCOOR′, OCONHR′, OCON(R′)₂, CONHR′, and CON(R′)₂, wherein each of the R′ groups is independently selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group; and even more preferred the substituent is OH. Most preferred R₁ is a substituted alkyl group having 8, 9, 10, 11, 12, or 13 carbon atoms; being 2 -hydroxy-1,3,5-trimethylhexyl and 2-hydroxy-1,3,5,7-tetramethyloctyl the most preferred.

Particularly preferred R₂ is selected from hydrogen, —CH₂CONHR₁₆, and —CH(OR₁₇)CONHR₁₈, wherein R₁₆ and R₁₈ are each independently selected from hydrogen and substituted or unsubstituted C₁-C₁₂ alkyl, and R₁₇ is selected from hydrogen, substituted or unsubstituted C₁-C₆ alkyl, CORₐ, and COORₐ, wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R₁₇ is hydrogen. More preferably R₁₆ and R₁₈ are each independently selected from hydrogen and substituted or unsubstituted C₁-C₆ alkyl. Even more preferably R₁₆ and R₁₈ are each independently selected from hydrogen, methyl, ethyl, n-propyl, iso-propyl and butyl, including n-butyl, tert-butyl, sec-butyl and iso-butyl; being hydrogen the most preferred group.

Particularly preferred R₃ is selected from —CH₂CH₂CONHR₁₉ and —CH(OR₂O)CH₃ wherein R₁₉ is selected from hydrogen and substituted or unsubstituted C₁-C₁₂ alkyl, and R₂₀ is selected from hydrogen, substituted or unsubstituted C₁-C₆ alkyl, CORₐ, and COORₐ, wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R₂₀ is hydrogen. More preferably R₁₉ is selected from hydrogen and substituted or unsubstituted C₁-C₆ alkyl. Even more preferably R₁₉ is selected from hydrogen, methyl, ethyl, n-propyl, iso-propyl and butyl, including n-butyl, tert-butyl, sec-butyl and iso-butyl; being hydrogen the most preferred group.

Particularly preferred R₄, R₅, and R₈ are each independently selected from hydrogen, substituted or unsubstituted C₁-C₆ alkyl, CORₐ, and COORₐ, wherein Rₐ is selected from hydrogen and substituted or unsubstituted C₁-C₁₂ alkyl. Particularly preferred Rₐ is substituted or unsubstituted C₁-C₆ alkyl; and even more preferred is methyl, ethyl, n-propyl, iso-propyl and butyl, including n-butyl, tert-butyl, sec-butyl and iso-butyl. More preferably R₄, R₅, and R₈ are hydrogen.

Particularly preferred R₆ and R₁₄ are each independently selected from hydrogen and substituted or unsubstituted C₁-C₁₂ alkyl. More preferably R₆ and R₁₄ are each independently selected from hydrogen and substituted or unsubstituted C₁-C₆ alkyl. Even more preferably R₆ and R₁₄ are each independently selected from hydrogen, methyl, ethyl, n-propyl, iso-propyl and butyl, including n-butyl, tert-butyl, sec-butyl and iso-butyl; being hydrogen the most preferred group.

Preferably R₆, R₁₄, R₁₆, R₁₈, and R₁₉ have the same meaning in the compounds of the invention.

Preferably R₄, R₅, R₈, R₁₇, and R₂₀ have the same meaning in the compounds of the invention.

Particularly preferred R₇ is a substituted or unsubstituted C₁-C₆ alkyl, which may be branched or unbranched. More preferred alkyl groups, which may be branched or unbranched, are those having 1, 2, 3, 4 or 5 carbon atoms; being methyl and 1,2-dimethyl-propyl the most preferred.

Particularly preferred R₉ and R₁₀ are each independently selected from hydrogen, substituted or unsubstituted C₁-C₁₂ alkyl, CORₐ, CONRₐR_{b}, and C(═NRₐ)NRₐR_{b}, wherein Rₐ and R_{b} are each independently selected from hydrogen and substituted or unsubstituted C₁-C₁₂ alkyl. Particularly preferred Rₐ and R_{b} are each independently selected from hydrogen and substituted or unsubstituted C₁-C₆ alkyl; and even more preferred are each independently selected from hydrogen, methyl, ethyl, n-propyl, iso-propyl and butyl, including n-butyl, tert-butyl, sec-butyl and iso-butyl. More preferably R₉ and R₁₀ are hydrogen.

Particularly preferred R₁₁ and R₁₃ are each independently selected from substituted or unsubstituted C₁-C₆ alkyl, which may be branched or unbranched. More preferred alkyl groups, which may be branched or unbranched, are those having 1, 2, 3, or 4 carbon atoms; being methyl and ethyl the most preferred. Preferably R₁₁ and R₁₃ have different meaning in the compounds of the invention.

Particularly preferred R₁₂ and R₁₅ are each independently selected from NRₐR_{b} and OR_{c}, wherein R_{c} is preferably selected from hydrogen, substituted or unsubstituted C₁-C₆ alkyl, CORₐ, and COORₐ, and wherein Rₐ and R_{b} are each independently selected from hydrogen and substituted or unsubstituted C₁-C₁₂ alkyl. Particularly preferred Rₐ and R_{b} are each independently selected from hydrogen and substituted or unsubstituted C₁-C₆ alkyl; and even more preferred are each independently selected from hydrogen, methyl, ethyl, n-propyl, iso-propyl and butyl, including n-butyl, tert-butyl, sec-butyl and iso-butyl. More preferably R_{c} is hydrogen. Preferably R₁₂ and R₁₅ are selected from OH and NH₂ and have the same meaning in the compounds of the invention.

In additional preferred embodiments, the preferences described above for the different substituents are combined. The present invention is also directed to such combinations of preferred substitutions in the formula (I) above.

In the present description and definitions, when there are several groups Rₐ, R_{b}, or R_{c} present in the compounds of the invention, and unless it is stated explicitly so, it should be understood that they can be each independently different within the given definition, i.e. Rₐ does not represent necessarily the same group simultaneously in a given compound of the invention.

Particularly preferred compounds of the invention are the following:

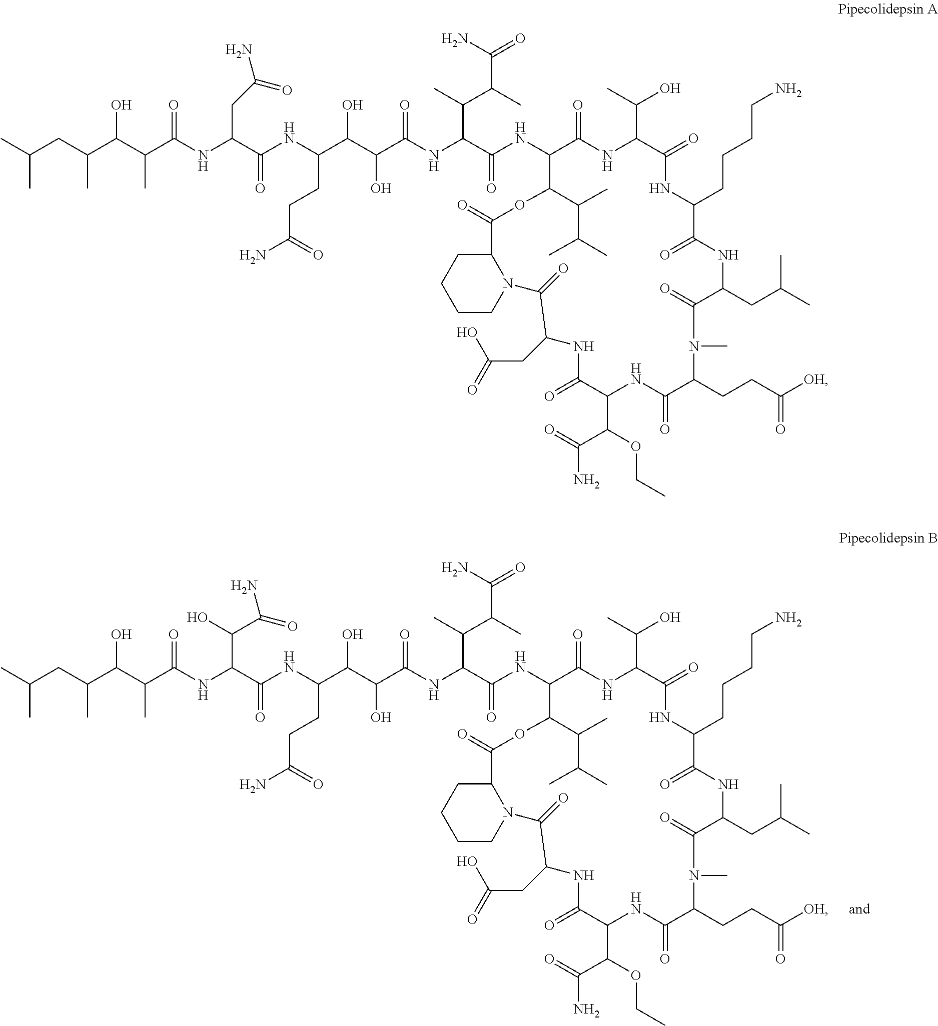

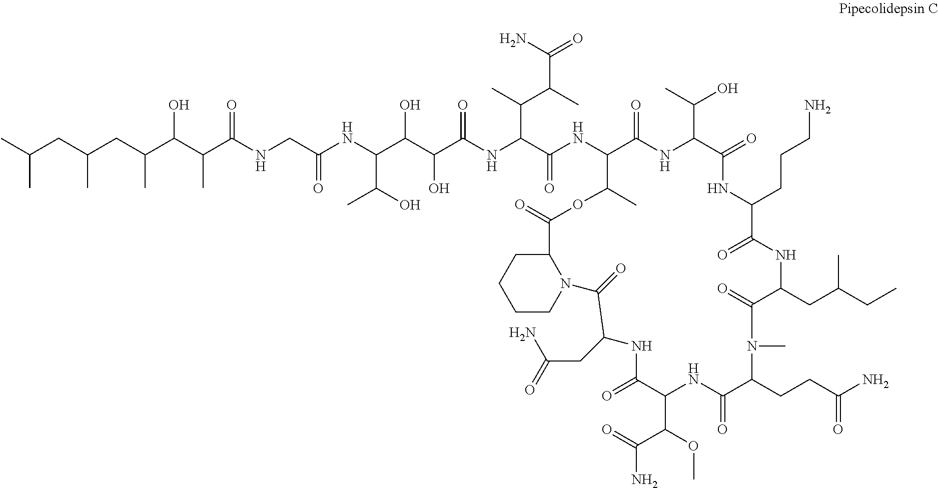

or pharmaceutically acceptable salts, tautomers, prodrugs or stereoisomers thereof.

Pipecolidepsins A, B and C were isolated from a sponge of the order Lithistida, family Neopeltidae, genus Homophymia, species Homophymia lamellosa Vacelet & Vasseur, 1971. This sponge was collected by hand using SCUBA diving in Saint Marie Island, Madagascar (17° 07. 436′ S/49° 47. 525′ E) at depths ranging between 3 and 7 m.

Description of this sponge: Thickly lamellar sponge (6 cm high, 10 cm wide, 2 cm thick) with a rounded outline consisting of several more or less individualized (especially at the top) but fused, with separate small oscules (2-3 mm diameter) situated at the top. Megascleras: Pseudophyllotrianes measuring 200-420 μm. Desmas, about 250-350 μm in size, resemble tetraclones but are monocrepidial. Other choanosomal megascleres are large styles to strongyles/tylotes measuring 380-560 μm long and 2.5-5 μm thick. Microscleres: are microspines amphiaster with long slender rays and are very abundant at the surface of the axial cavity and choanosomal canals; they measure 12.2-16.25 μm long and 6.25-12.5 μm wide.

Geographical distribution of this sponge: Madagascar, New Zealand, Reunion Island.

Additionally, compounds of the invention can be obtained by modifying those already obtained from the natural source or by further modifying those already modified by using a variety of chemical reactions. Thus, hydroxyl groups can be acylated by standard coupling or acylation procedures, for instance by using acetic acid, acetyl chloride or acetic anhydride in pyridine or the like. Formate groups can be obtained by heating hydroxyl precursors in formic acid. Carbamates can be obtained by heating hydroxyl precursors with isocyanates. Hydroxyl groups can be converted into halogen groups through intermediate sulfonates for iodide, bromide or chloride, or directly using a sulfur trifluoride for fluorides; or they can be reduced to hydrogen by reduction of intermediate sulfonates. Hydroxyl groups can also be converted into alkoxy groups by alkylation using an alkyl bromide, iodide or sulfonate, or into amino lower alkoxy groups by using, for instance, a protected 2-bromoethylamine. Amido groups can be alkylated or acylated by standard alkylation or acylation procedures, for instance by using, respectively, KH and methyl iodide or acetyl chloride in pyridine or the like. Ester groups can be hydrolized to carboxylic acids or reduced to aldehyde or to alcohol. Carboxylic acids can be coupled with amines to provide amides by standard coupling or acylation procedures. When necessary, appropriate protecting groups can be used on the substituents to ensure that reactive groups are not affected. The procedures and reagents needed to prepare these derivatives are known to the skilled person and can be found in general textbooks such as March's Advanced Organic Chemistry 6th Edition 2007, Wiley Interscience.

An important feature of the above described compounds of formula I is their bioactivity and in particular their cytotoxic activity against tumor cells. Thus, with this invention we provide pharmaceutical compositions of compounds of general formula I, or a pharmaceutically acceptable salt, tautomer, prodrug or stereoisomer thereof, that possess cytotoxic activities and their use as anticancer agents. The present invention further provides pharmaceutical compositions comprising a compound of general formula I, or a pharmaceutically acceptable salt, tautomer, prodrug or stereoisomer thereof, with a pharmaceutically acceptable carrier or diluent.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

As used herein, the terms “treat”, “treating” and “treatment” include the eradication, removal, modification, or control of a tumor or primary, regional, or metastatic cancer cells or tissue and the minimization or delay of the spread of cancer.

The compounds of the invention have anticancer activity against several cancers types which include, but are not limited to, lung cancer, colon cancer, and breast cancer.

Thus, in alternative embodiments of the present invention, the pharmaceutical composition comprising the compounds of formula (I) as defined above is for the treatment of lung cancer, colon cancer or breast cancer.

### EXAMPLES

### Example 1

### Description of the Marine Organism and Collection Site

Homophymia lamellosa Vacelet & Vasseur, 1971 was collected by hand using SCUBA diving in Saint Marie Island, Madagascar (17° 07. 436′ S/49° 47. 525′ E) at depths ranging between 3 and 7 m.

### Example 2

### Isolation of Pipecolidepsin A

The frozen specimen of Example 1 (82 g) was diced and extracted with H₂O (3×300 mL) and then with a mixture of CH₃OH:CH₂Cl₂ (50:50, 3×300 mL) at room temperature. The combined aqueous and organic extracts were concentrated separately to yield residues of 2.82 g and 700 mg, respectively.

The aqueous extract was subjected to VLC on Lichroprep RP-18 with a stepped gradient from H₂O to CH₃OH. The fraction eluted with CH₃OH:H₂O (3:1, 58.7 mg) was subjected to semipreparative reversed phase HPLC (SymmetryPrep C₁₈, 7 μm, 7.8×150 mm, gradient H₂O+0.1% TFA:CH₃CN+0.1% TFA from 25 to 40% CH₃CN in 22 min and then from 40 to 100% in 6 min, UV detection, flow 2.5 mL/min). A fraction with a retention time from 20 to 26 min of this chromatography was further purified by semipreparative reversed phase HPLC (Atlantis dC₁₈, 10 μm, 10×150 mm, isocratic H₂O+0.1% TFA:CH₃CN+0.1% TFA (64:36), UV detection, flow 2.5 mL/min), to yield Pipecolidepsin A (3 mg, retention time: 31.45 min).

The organic extract was subjected to VLC on Lichroprep RP-18 with a stepped gradient from H₂O to CH₃OH. The fraction eluted with CH₃OH:H₂O (3:1, 14.1 mg) was subjected to semipreparative reversed phase HPLC (SymmetryPrep C₁₈ 7 μm, 7.8×150 mm, gradient H₂O+0.1% TFA:CH₃CN+0.1% TFA from 22 to 42% CH₃CN in 25 min and then from 42 to 100% in 7 min, UV detection, flow 2.5 mL/min) to yield a further amount of Pipecolidepsin A (2 mg, retention time: 23.84 min).

Pipecolidepsin A: Amorphous white solid. (+)HRMALDIMS m/z 1655.90918 [M+H]⁺ (calc. for C₇₄H₁₂₇N₁₆O₂₆, 1655.91020). ¹H (500 MHz) and ¹³C NMR (125 MHz) see Table 1.

TABLE 1
¹H and ¹³C NMR data of Pipecolidepsin A (CD₃OH)

| | | |
|---|---|---|
| N^{o} | ¹³C, mult | ¹H (Multiplicity, J) |

Pip

| | | |
|---|---|---|
| 1 | 170.4, s | — |
| 2 | 53.9, d | 5.27 (m) |
| 3 | 27.6, t | 2.19 (m) |
| | | 1.64 (m) |
| 4 | 22.6, t | 1.74 (m) |
| | | 1.25 (m) |
| 5 | 27.9, t | 1.61 (m) |
| | | 1.58 (m) |
| 6 | 44.6, t | 3.70 (m) |
| | | 3.12 (m) |

Asp

| | | |
|---|---|---|
| 1 | 170.5, s | 8.39 (d, 9.0) NH |
| 2 | 47.2, d | 5.34 (m) |
| 3 | 36.5, t | 2.90 (dd, 17.0, 8.0) |
| | | 2.46 (dd, 16.5, 4.0) |
| 4 | 173.8, s | — |

EtOAsn

| 1 | 169.7, s | 6.49 (d, 9.0) NH | |
|---|---|---|---|
| 2 | 56.7, d | 4.92 (m) | |
| 3 | 78.5, d | 4.62 (br s) | |
| 4 | 174.1, s | 7.53 (br s) | NH₂ |
| | | 7.31 (br s) | |

| | | |
|---|---|---|
| OCH₂ | 68.0, t | 3.67 (m) |
| | | 3.48 (m) |
| CH₃ | 16.0, q | 1.19 (t, 7.0) |

MeGlu

| | | |
|---|---|---|
| 1 | 172.2, s | — |
| 2 | 58.2, d | 5.42 (d, 8.0) |
| 3 | 23.5, t | 2.46 (m) |
| | | 1.81 (m) |
| 4 | 31.9, t | 2.19 (m) |
| | | 2.08 (m) |
| 5 | 177.8, s | — |
| NMe | 31.4, q | 2.96 (s) |

Leu

| | | |
|---|---|---|
| 1 | 177.0, s | 7.60 (d, 6.0) NH |
| 2 | 51.3, d | 4.58 (m) |
| 3 | 38.9, t | 2.39 (m) |
| | | 1.55 (m) |
| 4 | 26.1, d | 2.03 (m) |
| 4-Me | 20.8, q | 1.03 (d, 6.5) |
| 5 | 24.1, q | 1.09^{a} |

Lys

| | | |
|---|---|---|
| 1 | 174.4, s | 7.85 (br d, 8.5) NH |
| 2 | 53.1, d | 4.49 (m) |
| 3 | 30.4, t | 2.02 (m) |
| | | 1.55 (m) |
| 4 | 23.5, t | 1.48 (m) |
| | | 1.36 (m) |
| 5 | 26.3, t | 1.66 (m) |
| 6 | 41.0, t | 2.95 (m) |
| NH₂ | — | Not observed |

Thr

| | | |
|---|---|---|
| 1 | 171.8, s | 8.33 (br s) NH |
| 2 | 64.3, d | 3.82 (br s) |
| 3 | 67.6, d | 4.39 (m) |
| 4 | 20.1, q | 1.34 (d, 6.0) |

AHDMHA

| | | |
|---|---|---|
| 1 | 174.6, s | 8.96 (d, 10.0) NH |
| 2 | 55.6, d | 5.29 (m) |
| 3 | 77.2, d | 5.62 (dd, 11.0, 2.0) |
| 4 | 39.1, d | 1.91 (m) |
| 4-Me | 8.8, q | 0.74 (d, 7.5) |
| 5 | 27.8, d | 1.93 (m) |
| 5-Me | 21.3, q | 0.94 (d, 6.5) |
| 6 | 15.3, q | 0.73 (d, 7.5) |

DiMeGln

| 1 | 174.6, s | 9.00 (d, 4.5) NH | |
|---|---|---|---|
| 2 | 59.0, d | 4.33 (dd, 11.0, 5.5) | |
| 3 | 37.2, d | 2.30 (m) | |
| 3-Me | 14.3, q | 1.09^{a} | |
| 4 | 42.5, d | 2.75 (dddd, 7.0, 2.5) | |
| 4-Me | 14.4, q | 1.24 (d, 7.0) | |
| 5 | 180.2, s | 7.45, (br s) | NH₂ |
| | | 6.94, (br s) | |

DADHOHA

| 1 | 176.4, s | 7.66 (d, 9.0) NH | |
|---|---|---|---|
| 2 | 72.9, d | 3.84 (m) | |
| 3 | 75.7, d | 3.59 (m) | |
| 4 | 51.1, d | 4.10 (m) | |
| 5 | 28.5, t | 1.92 (m) | |
| | | 1.78 (m) | |
| 6 | 32.6, t | 2.23 (m) | |
| 7 | 178.6, s | 7.53, (br s) | NH₂ |
| | | 6.83, (br s) | |

Asn

| 1 | 174.8, s | 8.35 (d, 8.0) NH | |
|---|---|---|---|
| 2 | 51.7, d | 4.69 (dd, 12.0, 6.5) | |
| 3 | 36.6, t | 2.92 (m) | |
| | | 2.84 (dd, 17.0, 5.0) | |
| 4 | 174.7, s | 7.21 (br s) | NH₂ |
| | | 6.75 (br s) | |

HTMHA

| | | |
|---|---|---|
| 1 | 179.1, s | — |
| 2 | 44.9, d | 2.62 (dq, 9.5, 7.0) |
| 2-Me | 14.4, q | 1.07^{a} |
| 3 | 79.6, d | 3.57 (m) |
| 4 | 33.5, d | 1.74 (m) |
| 4-Me | 17.4, q | 0.99 (d, 6.5) |
| 5 | 39.3. t | 1.16 (m) |
| 6 | 26.2, d | 1.66 (m) |
| 6-Me | 21.5, q | 0.87 (d, 6.5) |
| 7 | 24.7, q | 0.94 (d, 6.5) |

^{a}Overlapped
HTMHA: 3-hydroxy-2,4,6-trimethylheptanoic acid;
DADHOHA: 4,7-diamino-2,3-dihydroxy-7-oxoheptanoic acid;
AHDMHA: 2-amino-3-hydroxy-4,5-dimethylhexanoic acid.

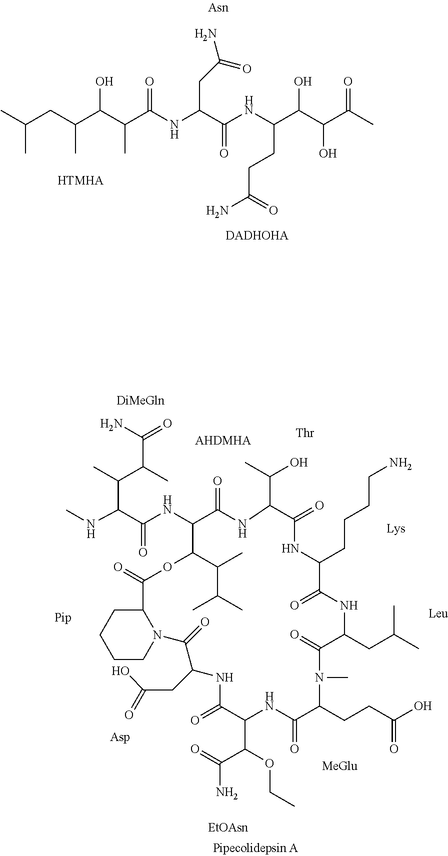

### Example 3

### Isolation of Pipecolidepsin B and C

A second group of specimens of Example 1 (382.5 g) was triturated and exhaustively extracted with 2-propanol (4×400 mL, 2×300 mL). The combined extracts were concentrated to yield a crude of 13.19 g. This crude was dissolved in 300 mL of H₂O and extracted with Hexane (3×300 mL), EtOAc (3×300 mL) and n-Butanol (3×100 mL).

The n-Butanol extract was evaporated to yield a crude of 5.86 g that was subjected to VLC on Lichroprep RP-18 with a stepped gradient from H₂O to CH₃OH and then CH₃OH:CH₂Cl₂ (50:50). Fractions eluted with CH₃OH:H₂O (75:25) and CH₃OH:H₂O (85:15) were pooled to give a fraction of 592.5 mg that was subjected to RP-18 column chromatography with a stepped gradient from H₂O:CH₃OH (35:65) to CH₃OH. Fractions eluted with H₂O:CH₃OH (30:70, 223.4 mg) were subjected to preparative HPLC (Symmetry C₁₈, 7 μm, 19×150 mm, gradient H₂O+0.1% TFA:CH₃CN+0.1% TFA from 22 to 42% CH₃CN in 25 min and then from 42 to 100% in 7 min, flow: 15 mL/min, UV detection) to yield a fraction containing a mixture of Pipecolidepsin A and B (retention time from 24.2 to 26.2 min). This fraction was further purified by semipreparative HPLC (X-Bridge Prep C₁₈, 5 μm, 10×150 mm, isocratic H₂O+0.1% TFA:CH₃CN+0.1% TFA (65:35), flow: 2.3 mL/min, UV detection) to obtain impure Pipecolidepsin A (39.9 mg, retention time: 26.49 min) and pure Pipecolidepsin B (13.6 mg, retention time: 24.99 min). Final purification of Pipecolidepsin A (14.9 mg) was achieved by semipreparative HPLC (Kromasil 100 C₈, 10 μm, 10×150 mm, gradient H₂O:CH₃CN from 30 to 45% in 30 min, flow: 2.5 mL/min, UV detection, retention time: 20.70 min).

Fractions from VLC on Lichroprep RP-18 eluted with CH₃OH and CH₃OH:CH₂Cl₂ (50:50) were pooled and subjected to preparative HPLC (Symmetry C₁₈, 7 μm, 19×150 mm, gradient H₂O+0.1% TFA:CH₃CN+0.1% TFA from 22 to 42% CH₃CN in 25 min and then from 42 to 100% in 7 min, flow: 15 mL/min, UV detection) to yield pure Pipecolidepsin C (18.9 mg) in the form of its trifluoroacetate salt.

Pipecolidepsin B: amorphous white solid. (+)HRMALDIMS m/z 1671.89954 [M+H]⁺ (calc. for C₇₄H₁₂₇N₁₆O₂₇, 1671.90511). ¹H (500 MHz) and ¹³C NMR (125 MHz) see Table 2.

Pipecolidepsin C: amorphous white solid. (+)HRMALDIMS m/z 1541.87463 [M+H]⁺ (calc. for C₆₉H₁₂₁N₁₆O₂₃, 1541.87850). ¹H (500 MHz) and ¹³C NMR (125 MHz) see Table 3.

TABLE 2
¹H and ¹³C NMR data of Pipecolidepsin B (CD₃OH)

| | | |
|---|---|---|
| N^{o} | ¹³C, mult | ¹H (Multiplicity, J) |

Pip

| | | |
|---|---|---|
| 1 | &^{b} | — |
| 2 | 53.9, d | 5.26 (m) |
| 3 | 27.6, t | 2.20 (m) |
| | | 1.63 (m) |
| 4 | 22.6, t | 1.75 (m) |
| | | 1.23 (m) |
| 5 | 28.0, t | 1.62 (m) |
| | | 1.56 (m) |
| 6 | 44.6, t | 3.69 (m) |
| | | 3.12 (m) |

Asp

| | | |
|---|---|---|
| 1 | &^{b} | 8.43 (d, 7.6) NH |
| 2 | 47.3, d | 5.32 (m) |
| 3 | 36.6, t | 2.85 (m) |
| | | 2.46 (m) |
| 4 | &^{b} | — |

EtOAsn

| | | |
|---|---|---|
| 1 | 169.8, s | 6.48 (d, 8.5) NH |
| 2 | 56.8, d | 4.91^{a} |
| 3 | 78.5, d | 4.60 (br s) |
| 4 | &^{b} | $^{e} NH₂ |
| OCH₂ | 68.0, t | 3.67 (m) |
| | | 3.48 (m) |
| CH₃ | 15.9, q | 1.18 (t, 7.0) |

MeGlu

| | | |
|---|---|---|
| 1 | 172.3, s | — |
| 2 | 58.2, d | 5.43 (m) |
| 3 | 23.3, t | 2.48 (m) |
| | | 1.78 (m) |
| 4 | 31.7, t | 2.22 (m) |
| | | 2.12 (m) |
| 5 | 177.7, s | — |
| NMe | 31.4, q | 2.95 (s) |

Leu

| | | |
|---|---|---|
| 1 | 177.0, s | 7.57 (d, 5.5) NH |
| 2 | 51.2, d | 4.58 (m) |
| 3 | 38.9, t | 2.38 (m) |
| | | 1.55 (m) |
| 4 | 26.1, d | 2.04 (m) |
| 4-Me | 20.8, q | 1.01 (d, 6.5) |
| 5 | 24.0, q | 1.09^{c} |

Lys

| | | |
|---|---|---|
| 1 | &^{b} | 7.88 (br s) NH |
| 2 | 53.4, d | 4.44 (m) |
| 3 | 30.5, t | 2.02 (m) |
| | | 1.58 (m) |
| 4 | 23.6, t | 1.50 (m) |
| | | 1.35 (m) |
| 5 | 26.3, t | 1.62 (m) |
| 6 | 41.1, t | 2.95 (m) |
| NH₂ | — | Not observed |

Thr

| | | |
|---|---|---|
| 1 | 171.9, s | 8.39 (br s) NH |
| 2 | 64.2, d | 3.82 (br s) |
| 3 | 67.6, d | 4.38 (m) |
| 4 | 20.2, q | 1.33 (d, 6.5) |

AHDMHA

| | | |
|---|---|---|
| 1 | &^{b} | 8.91 (br d, 8.5) NH |
| 2 | 55.6, d | 5.29 (m) |
| 3 | 77.2, d | 5.57 (br d, 11.0) |
| 4 | 39.1, d | 1.93 (m) |
| 4-Me | 8.7, q | 0.73 (d, 7.0) |
| 5 | 27.9, d | 1.92 (m) |
| 5-Me | 21.3, q | 0.94 (d, 6.5) |
| 6 | 15.2, q | 0.71 (d, 7.5) |

DiMeGln

| | | |
|---|---|---|
| 1 | &^{b} | 9.19 (d, 4.5) NH |
| 2 | 59.0, d | 4.28 (dd, 10.5, 5.5) |
| 3 | 36.9, d | 2.31 (m) |
| 3-Me | 14.1^{f}, q | 1.05^{c} |
| 4 | 42.7, d | 2.76 (m) |
| 4-Me | 14.4, q | 1.23 (d, 7.0) |
| 5 | 180.6, s | $^{e} NH₂ |

DADHOHA

| | | |
|---|---|---|
| 1 | 176.3, s | 7.53^{d} NH |
| 2 | 72.8, d | 3.92 (m) |
| 3 | 75.6, d | 3.57 (m) |
| 4 | 51.0, d | 4.10 (m) |
| 5 | 28.5, t | 1.92 (m) |
| | | 1.75 (m) |
| 6 | 32.7, t | 2.20 (m) |
| 7 | 178.7, s | $^{e} NH₂ |

HOAsn

| | | |
|---|---|---|
| 1 | &^{b} | 8.28 (d, 7.0) NH |
| 2 | 58.2, d | 4.86^{a} |
| 3 | 72.2, d | 4.38 (m) |
| 4 | &^{b} | $^{e} NH₂ |

HTMA

| | | |
|---|---|---|
| 1 | 179.3, s | — |
| 2 | 44.9, d | 2.69 (dq, 9.0, 7.0) |
| 2-Me | 14.3^{f}, q | 1.06^{c} |
| 3 | 79.9, d | 3.55 (m) |
| 4 | 33.4, d | 1.75 (m) |
| 4-Me | 17.4, q | 0.98 (d, 7.0) |
| 5 | 39.1. t | 1.16 (m) |
| 6 | 26.2, d | 1.63 (m) |
| 6-Me | 21.5, q | 0.86 (d, 6.5) |
| 7 | 24.7, q | 0.93 (d, 6.5) |

^{a}Under solvent
^{b}Carbonyl signals at these positions & (δ_{c} 174.7, 174.4, 174.2, 173.8, 170.6, and 170.4 ppm) were not assigned due to the failure of obtaining ¹H— ¹³C long range connectivities. Three carbonyl signals were overlapped/not detected
^{c}Methyl signals overlapped
^{d}NH and NH₂ signals overlapped
^{e}Assignments of NH₂ at these positions $ (δ_{H} 7.54^{d}/6.81, 7.50/6.96, 7.53^{d}/7.30, and 7.16/
6.79 ppm) are interchangeable
^{f}Assignments can be interchanged
HTMHA: 3-hydroxy-2,4,6-trimethylheptanoic acid;
DADHOHA: 4,7-diamino-2,3-dihydroxy-7-oxoheptanoic acid;
AHDMHA: 2-amino-3-hydroxy-4,5-dimethylhexanoic acid.

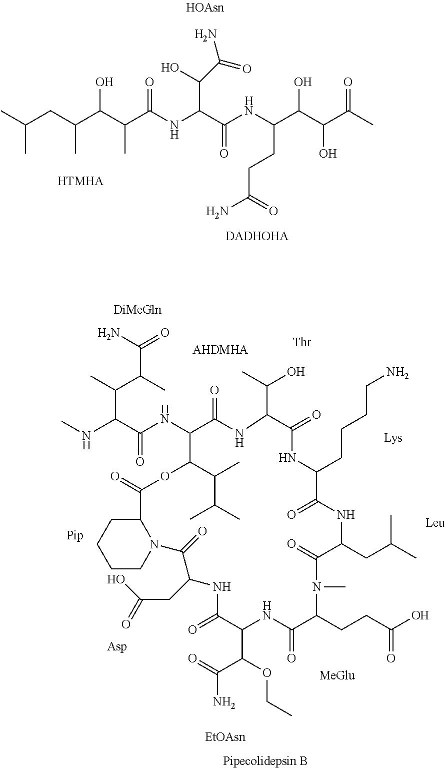

TABLE 3
¹H and ¹³C NMR data of Pipecolidepsin C (CD₃OH)

| | | |
|---|---|---|
| N^{o} | ¹³C, mult | ¹H (Multiplicity, J) |

Pip

| | | |
|---|---|---|
| 1 | 170.8, s | — |
| 2 | 53.7, d | 5.22 (br d, 3.5) |
| 3 | 27.4, t | 2.16 (m) |
| | | 1.60 (m) |
| 4 | 22.1, t | 1.70 (m) |
| | | 1.22 (m) |
| 5 | 26.2, t | 1.62 (m) |
| | | 1.49 (m) |
| 6 | 44.8, t | 3.65 (m) |
| | | 3.06 (m) |

Asn

| | | |
|---|---|---|
| 1 | 171.3, s | 8.31 (d, 9.5) NH |
| 2 | 47.4, d | 5.39 (m) |
| 3 | 37.7, t | 2.85 (m) |
| | | 2.38 (dd, 15.5, 5.0) |
| 4 | 175.5, s | $^{b} NH₂ |

MeOAsn

| 1 | &^{a} | 6.48 (d, 8.5) NH | |
|---|---|---|---|
| 2 | 56.8, d | 4.90^{c} | |
| 3 | 80.3, d | 4.45 (d, 2.0) | |
| 4 | 173.7, s | 7.31 (s) | NH₂ |
| | | 7.58 (s) | |

| | | |
|---|---|---|
| OMe | 59.6, q | 3.38, s |

MeGln

| | | |
|---|---|---|
| 1 | 172.4, s | — |
| 2 | 57.9, d | 5.41 (m) |
| 3 | 23.9, t | 2.37 (m) |
| | | 1.78 (m) |
| 4 | 32.4, t | 2.15 (m) |
| | | 2.06 (m) |
| 5 | 177.8, s | $^{b} NH₂ |
| NMe | 31.3, q | 2.92 (s) |

AMHA

| | | |
|---|---|---|
| 1 | 176.7, s | 7.53 (d, 7.0) NH |
| 2 | 50.5, d | 4.67 (ddd, 12.5, 7.0, 2.5) |
| 3 | 37.3, t | 2.24 (m) |
| | | 1.58 (m) |
| 4 | 31.8, d | 1.74 (m) |
| 4-Me | 17.8, q | 0.97 (d, 7.0) |
| 5 | 31.5, t | 1.44 (m) |
| | | 1.34 (m) |
| 6 | 11.6, q | 0.94 (t, 7.5) |

Orn

| | | |
|---|---|---|
| 1 | &^{a} | 8.27 (d, 9.5) NH |
| 2 | 52.3, d | 4.60 (m) |
| 3 | 27.9, t | 2.09 (m) |
| | | 1.53 (m) |
| 4 | 25.0, t | 1.72 (m) |
| | | 1.62 (m) |
| 5 | 40.3, t | 2.88 (m) |
| NH₂ | — | Not observed |

Thr-1

| | | |
|---|---|---|
| 1 | 171.9, s | 8.48 (br s) NH |
| 2 | 63.9, d | 3.94 (t, 2.5) |
| 3 | 67.4, d | 4.43 (qd, 6.5, 2.5) |
| 4 | 20.2, q | 1.33, d, 6.5 |

Thr-2

| | | |
|---|---|---|
| 1 | &^{a} | 8.97 (d, 10.0) NH |
| 2 | 55.6, d | 5.38, m |
| 3 | 71.6, d | 5.71 (qd, 7.0, 4.0) |
| 4 | 14.7, q | 1.22(d, 7.0) |

DiMeGln

| 1 | &a | 9.22 (d, 5.5) NH | |
|---|---|---|---|
| 2 | 59.4, d | 4.18 (dd, 10.5, 5.0) | |
| 3 | 37.7, d | 2.22 (m) | |
| 3-Me | 13.8, q | 1.06 (d, 7.5) | |
| 4 | 42.2, d | 2.71 (dq, 7.0, 3.5) | |
| 4-Me | 15.0, q | 1.22 (d, 7.0) | |
| 5 | 179.9, s | 6.95 | NH₂ |
| | | 7.44 (s) | |

ATHHA

| | | |
|---|---|---|
| 1 | &^{a} | 7.73 (d, 9.0) NH |
| 2 | 72.8^{d}, d | 3.88 (m) |
| 3 | 75.4^{d}, d | 3.88 (m) |
| 4 | 55.5, d | 4.03 (m) |
| 5 | 70.2, d | 4.04 (m) |
| 6 | 20.6, q | 1.16 (d, 5.5) |

Gly

| | | |
|---|---|---|
| 1 | 173.0, s | 8.40 (t, 6.0) NH |
| 2 | 44.1, t | 4.00 (dd, 16.5, 6.0) |
| | | 3.85 (dd, 16.5, 6.0) |

HTMNA

| | | |
|---|---|---|
| 1 | 179.6, s | — |
| 2 | 45.1, d | 2.57 (dq 9.0, 7.0) |
| 2-Me | 14.7, q | 1.08 (d, 7.0) |
| 3 | 79.3, d | 3.52 (dd, 9.0, 3.0) |
| 4 | 33.4, d | 1.73 (m) |
| 4-Me | 17.6, q | 0.95 (d, 7.5) |
| 5 | 38.8, t | 1.10 (m) |
| | | 1.25 (m) |
| 6 | 28.9, d | 1.56 (m) |
| 6-Me | 24.5, q | 0.89 (d, 7.0) |
| 7 | 46.7, t | 1.17 (m) |
| | | 0.88 (m) |
| 8 | 26.4, d | 1.67 (m) |
| 8-Me | 21.5, q | 0.89 (d, 7.0) |
| 9 | 21.8, q | 0.84 (d, 6.5) |

^{a}Carbonyl signals at these positions & (δ_{c} 176.0, 174.1, 173.9, 173.6, and 170.0 ppm) were not assigned due to the failure of obtaining ¹H—¹³C long range connectivities
^{b}Assignments of NH₂ at these positions $ (δ_{H} 6.75/7.22 and 6.76/7.54 ppm) are interchangeable
^{c}Under solvent
^{d}Assignments can be interchanged
HTMNA: 3-hydroxy-2,4,6,8-tetramethylnonanoic acid;
ATHHA: 4-amino-2,3,5-trihydroxyhexanoic acid;
AMHA: 2-amino-4-methylhexanoic acid.

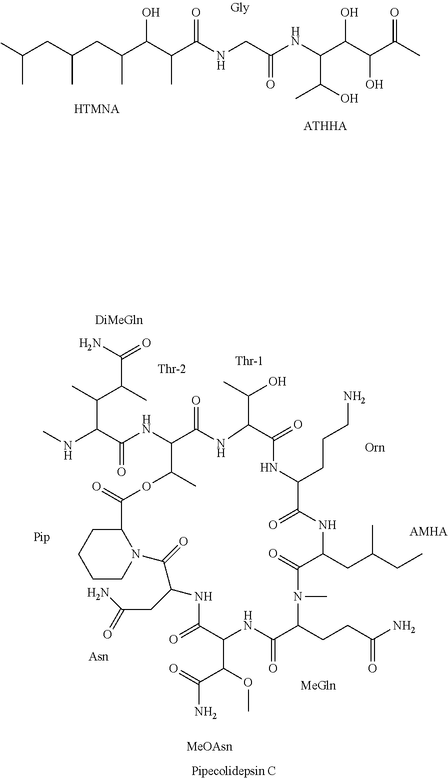

### Example 4

### Bioassays for the Detection of Antitumor Activity

The aim of this assay is to evaluate the in vitro cytostatic (ability to delay or arrest tumor cell growth) or cytotoxic (ability to kill tumor cells) activity of the samples being tested.

### Cell Lines

| Name | N° ATCC | Species | Tissue | Characteristics |
|---|---|---|---|---|
| A549 | CCL-185 | human | lung | lung carcinoma (NSCLC) |
| HT29 | HTB-38 | human | colon | colorectal |
| | | | | adenocarcinoma |
| MDA-MB-231 | HTB-26 | human | breast | breast adenocarcinoma |

### Evaluation of Cytotoxic Activity Using the SBR Colorimetric Assay

A colorimetric assay, using sulforhodamine B (SRB) reaction has been adapted to provide a quantitative measurement of cell growth and viability (following the technique described by Skehan et al. J. Natl. Cancer Inst. 1990, 82, 1107-1112).

This form of assay employs SBS-standard 96-well cell culture microplates (Faircloth et al. Methods in Cell Science, 1988, 11(4), 201-205; Mosmann et al, Journal of Immunological Methods, 1983, 65(1-2), 55-63). All the cell lines used in this study were obtained from the American Type Culture Collection (ATCC) and derive from different types of human cancer.

Cells were maintained in Dulbecco's Modified Eagle Medium (DMEM) supplemented with 10% Fetal Bovine Serum (FBS), 2 mM L-glutamine, 100 U/mL penicillin and 100 U/mL streptomycin at 37° C., 5% CO₂ and 98% humidity. For the experiments, cells were harvested from subconfluent cultures using trypsinization and resuspended in fresh medium before counting and plating.

Cells were seeded in 96 well microtiter plates, at 5×10³ cells per well in aliquots of 150 μL, and allowed to attach to the plate surface for 18 hours (overnight) in drug free medium. After that, one control (untreated) plate of each cell line was fixed (as described below) and used for time zero reference value. Culture plates were then treated with test compounds (50 μL aliquots of 4× stock solutions in complete culture medium plus 4% DMSO) using ten serial dilutions (concentrations ranging from 10 to 0.00262 μg/mL) and triplicate cultures (1% final concentration of DMSO). After 72 hours treatment, the antitumor effect was measured by using the SRB methodology: Briefly, cells were washed twice with PBS, fixed for 15 min in 1% glutaraldehyde solution at room temperature, rinsed twice in PBS, and stained in 0.4% SRB solution for 30 min at room temperature. Cells were then rinsed several times with 1% acetic acid solution and air-dried at room temperature. SRB was then extracted in 10 mM trizma base solution and the absorbance measured in an automated spectrophotometric plate reader at 490 nm. Effects on cell growth and survival were estimated by applying the NCI algorithm (Boyd M R and Paull K D. Drug Dev. Res. 1995, 34, 91-104).

Using the mean±SD of triplicate cultures, a dose-response curve was automatically generated using nonlinear regression analysis. Three reference parameters were calculated (NCI algorithm) by automatic interpolation: GI₅₀=compound concentration that produces 50% cell growth inhibition, as compared to control cultures; TGI=total cell growth inhibition (cytostatic effect), as compared to control cultures, and LC₅₀=compound concentration that produces 50% net cell killing (cytotoxic effect).

Table 4 illustrates data on the biological activity of compounds of the present invention.

TABLE 4
Cytotoxicity assay-Activity Data (Molar) of Pipecolidepsin
A, B and C.

| | | Pipecolidepsin | Pipecolidepsin | |
|---|---|---|---|---|
| | | A | B | Pipecolidepsin C |
| MDA-MB- | GI₅₀ | 7.25E−07 | 2.33E−08 | 4.80E−07 |
| 231 | TGI | 1.15E−06 | 6.58E−08 | 6.48E−07 |
| | LC₅₀ | 1.75E−06 | 2.09E−07 | 9.07E−07 |
| HT29 | GI₅₀ | 1.15E−06 | 1.44E−08 | 6.48E−07 |
| | TGI | 1.33E−06 | 2.69E−08 | 8.43E−07 |
| | LC₅₀ | 1.57E−06 | 6.58E−08 | 1.04E−06 |
| A549 | GI₅₀ | 6.04E−07 | 3.53E−08 | 4.28E−07 |
| | TGI | 9.06E−07 | 1.08E−07 | 5.25E−07 |
| | LC₅₀ | 1.39E−06 | 3.53E−07 | 6.48E−07 |

1. A compound of general formula I 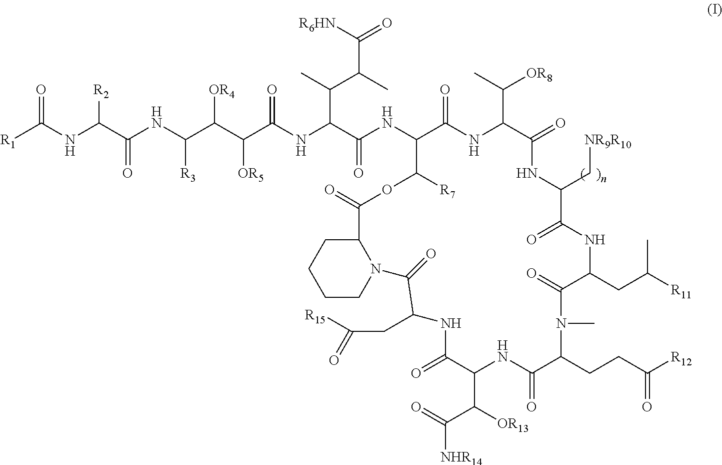
wherein
R₁ is selected from substituted or unsubstituted C₁-C₁₈ alkyl, substituted or unsubstituted C₂-C₁₈ alkenyl, substituted or unsubstituted C₂-C₁₈ alkynyl, substituted or unsubstituted aryl, and substituted or unsubstituted heterocyclic group;
R₂ is selected from hydrogen, —CH₂CONHR₁₆, and —CH(OR₁₇)CONHR₁₈;
R₃ is selected from —CH₂CH₂CONHR₁₉ and —CH(OR₂₀)CH₃;
each R₄, R₅, R₈, R₁₇, and R₂₀ is independently selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl;
each R₆, R₁₄, R₁₆, R₁₈, and R₁₉ is independently selected from hydrogen, CORₐ, COORₐ, CONRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₇, R₁₁, and R₁₃ is independently selected from substituted or unsubstituted C₁-C₁₂ alkyl;
each R₉ and R₁₀ is independently selected from hydrogen, CORₐ, COORₐ, CONRₐR_{b}, C(═NRₐ)NRₐR_{b}, substituted or unsubstituted C₁-C₁₂ alkyl, substituted or unsubstituted C₂-C₁₂ alkenyl, and substituted or unsubstituted C₂-C₁₂ alkynyl;
each R₁₂ and R₁₅ is independently selected from OR_{c}, NRₐR_{b}, CORₐ, NRₐCONRₐR_{b}, NRₐC(═NRₐ)NRₐR_{b}, halogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
n is 3 or 4;
R_{c} is selected from hydrogen, CORₐ, COORₐ, CONRₐR_{b}, SO₂Rₐ, SO₃Rₐ, substituted or unsubstituted C₁-C₁₂ alkyl, substituted or unsubstituted C₂-C₁₂ alkenyl, and substituted or unsubstituted C₂-C₁₂ alkynyl; and
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
or a pharmaceutically acceptable salt, tautomer, prodrug or stereoisomer thereof.

2. The compound according to claim 1, wherein R₁ is selected from substituted or unsubstituted C₁-C₁₈ alkyl and substituted or unsubstituted C₂-C₁₈ alkenyl, which may be branched or unbranched.

3. The compound according to claim 1, wherein R₁ is selected from substituted C₇-C₁₄ alkyl and substituted C₇-C₁₄ alkenyl, wherein they are independently substituted by one or more substituents selected from OR′, OSO₂R′, OSO₃R′, halogen, OCOR′, OCOOR′, OCONHR′, OCON(R′)₂, CONHR′, and CON(R′)₂, wherein each of the R′ groups is independently selected from the group consisting of hydrogen, substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group.

4. The compound according to claim 1, wherein R₁ is selected from 2-hydroxy-1,3,5-trimethylhexyl and 2-hydroxy-1,3,5,7-tetramethyloctyl.

5. The compound according to claim 1, wherein R₂ is selected from hydrogen, —CH₂CONHR₁₆, and —CH(OR₁₇)CONHR₁₈, and wherein R₁₆ and R₁₈ are each independently selected from hydrogen and substituted or unsubstituted C₁-C₆ alkyl, and R₁₇ is selected from hydrogen, substituted or unsubstituted C₁-C₆ alkyl, CORₐ, and COORₐ, wherein Rₐ is substituted or unsubstituted C₁-C₆ alkyl.

6. The compound according to claim 5, wherein R₂ is hydrogen.

7. The compound according to claim 5, wherein R₂ is —CH₂CONHR₁₆ and wherein R₁₆ is selected from hydrogen and substituted or unsubstituted C₁-C₆ alkyl.

8. The compound according to claim 5, wherein R₂ is —CH(OR₁₇)CONHR₁₈ and wherein R₁₇ is selected from hydrogen, substituted or unsubstituted C₁-C₆ alkyl, CORₐ, and COORₐ, wherein Rₐ is substituted or unsubstituted C₁-C₆ alkyl, and R₁₈ is selected from hydrogen and substituted or unsubstituted C₁-C₆ alkyl.

9. The compound according to claim 5, wherein R₂ is selected from hydrogen, —CH₂CONH₂, and —CH(OH)CONH₂.

10. The compound according to claim 1, wherein R₃ is selected from —CH₂CH₂CONHR₁₉ and —CH(OR₂₀)CH₃ wherein R₁₉ is selected from hydrogen and substituted or unsubstituted C₁-C₆ alkyl, and R₂₀ is selected from hydrogen, substituted or unsubstituted C₁-C₆ alkyl, CORₐ, and COORₐ, wherein Rₐ is substituted or unsubstituted C₁-C₆ alkyl.

11. The compound according to claim 10, wherein R₃ is selected from —CH₂CH₂CONH₂ and —CH(OH)CH₃.

12. The compound according to claim 1, wherein R₄, R₅, and R₈ are each independently selected from hydrogen, substituted or unsubstituted C₁-C₆ alkyl, CORₐ, and COORₐ, wherein Rₐ is substituted or unsubstituted C₁-C₆ alkyl.

13. The compound according to claim 12, wherein R₄, R₅, and R₈ are hydrogen.

14. The compound according to claim 1, wherein R₆ and R₁₄ are each independently selected from hydrogen and substituted or unsubstituted C₁-C₆ alkyl.

15. The compound according to claim 14, wherein R₆ and R₁₄ are hydrogen.

16. The compound according to claim 1, wherein R₇ is a substituted or unsubstituted C₁-C₆ alkyl, which may be branched or unbranched.

17. The compound according to claim 16, wherein R₇ is selected from methyl and 1,2-dimethyl-propyl.

18. The compound according to claim 1, wherein R₉ and R₁₀ are each independently selected from hydrogen, substituted or unsubstituted C₁-C₁₂ alkyl, CORₐ, CONRₐR_{b} and C(═NRₐ)NRₐR_{b}, wherein Rₐ and R_{b} are each independently selected from hydrogen and substituted or unsubstituted C₁-C₆ alkyl.

19. The compound according to claim 18, wherein R₉ and R₁₀ are hydrogen.

20. The compound according to claim 1, wherein R₁₁ and R₁₃ arc each independently selected from substituted or unsubstituted C₁-C₆ alkyl.

21. The compound according to claim 20, wherein R₁₁ and R₁₃ are each independently selected from methyl and ethyl.

22. The compound according to claim 1, wherein R₁₂ and R₁₅ are each independently selected from NRₐR_{b} and OR_{c}, wherein R_{c} is preferably selected from hydrogen, substituted or unsubstituted C₁-C₆ alkyl, CORₐ, and COORₐ, and wherein Rₐ and R_{b} are each independently selected from hydrogen and substituted or unsubstituted C₁-C₆ alkyl.

23. The compound according to claim 22, wherein R₁₂ and R₁₅ are selected from OH and NH₂.

24. The compound according to claim 1, wherein n is 3.

25. The compound according to claim 1, wherein n is 4.

26. The compound according to claim 1, having the following structure: 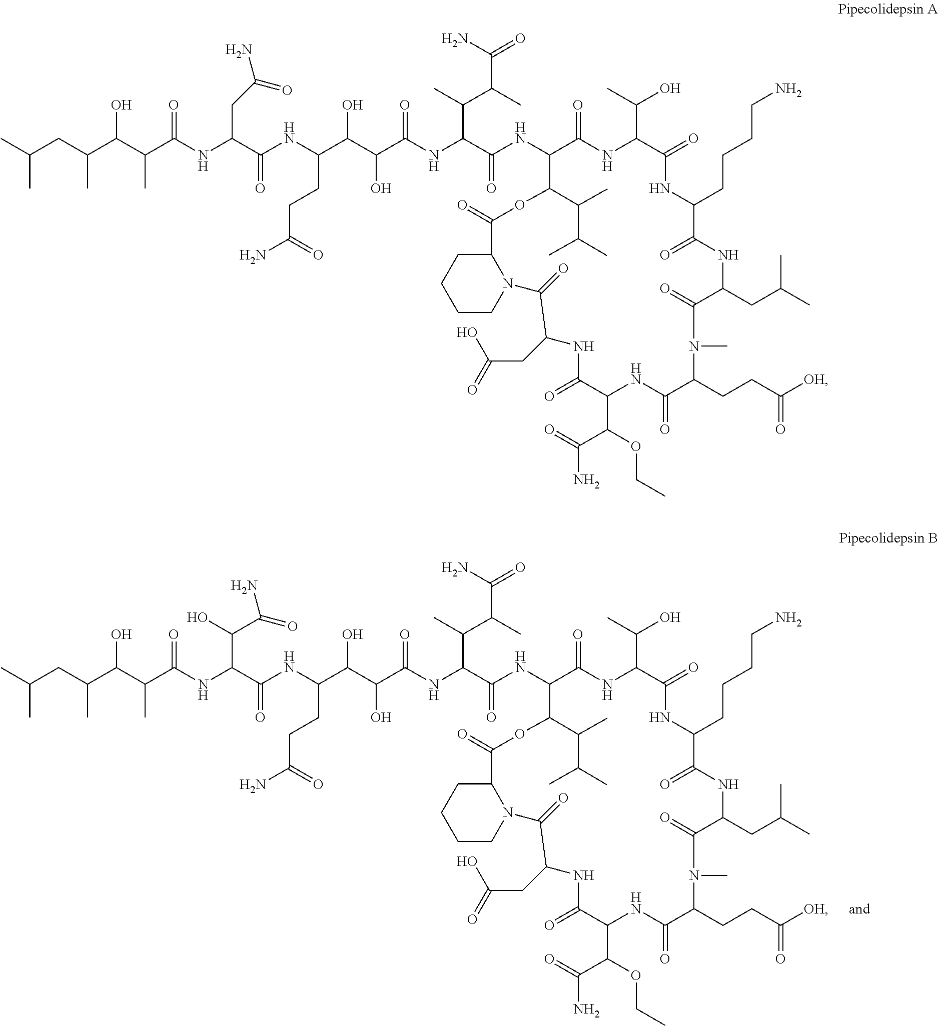 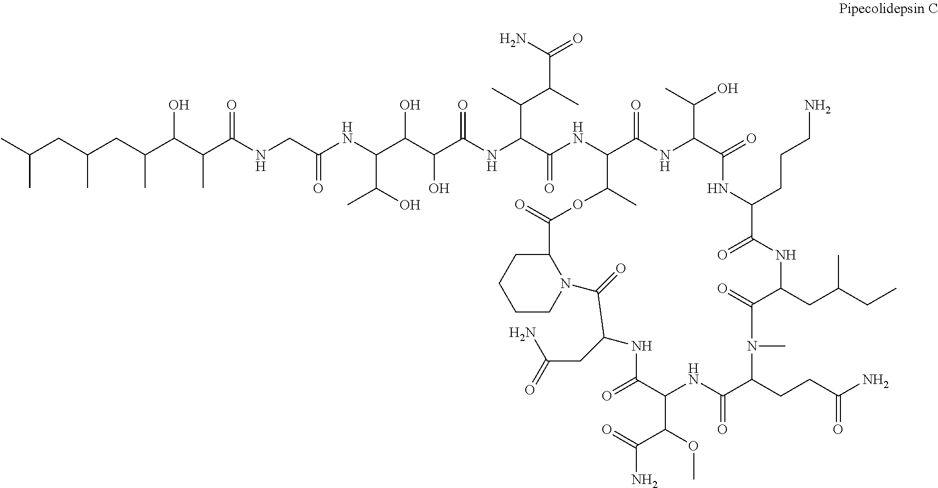
or a pharmaceutically acceptable salt, tautomer, prodrug or stereoisomer thereof.

27. A pharmaceutical composition comprising a compound according to claim 1, or a pharmaceutically acceptable salt, tautomer, prodrug or stereoisomer thereof, and a pharmaceutically acceptable carrier or diluent.

28. (canceled)

29. (canceled)

30. A method of treating a patient affected by cancer which comprises administering to said affected individual in need thereof a therapeutically effective amount of a compound as defined in claim 1.
